(19) United States
(12) Patent Application Publication
(10) Pub. No.: US 2010/0004679 A1
(43) Pub. Date: Jan. 7, 2010

(54) MEDICAL DEVICE

(75) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: Peter Osypka Stiftung Stiftung Des Burgerlichen Rechts, Grenzach-Wyhlen (DE)

(21) Appl. No.: 12/439,227

(22) PCT Filed: Aug. 9, 2007
(86) PCT No.: PCT/EP2007/007043

§ 371 Date: Feb. 27, 2009

(30) Foreign Application Priority Data
Sep. 25, 2006 (DE) 10 2006 045 545.2

(51) Int. Cl. A61B 17/03

(52) U.S. Cl. 606/213

(57) ABSTRACT

A medical device (1), in particular an occlusion instrument, for closing congenital or pathologically formed openings in the human body, with a collapsible braid (2) of wires and/or filaments made of nitinol or of similar material having a shape-memory effect, wherein the ends (3) of the wires or filaments of the braid are brought together by at least one closure assembly (4) and are held there. To ensure a secure connection of the free ends (3) of the wires of the braid (2), and to be able to easily handle the device and connect it quickly and reliably in a releasable manner to a positioning means, it is provided that the closure assembly (4) is formed by a sleeve into which the ends (3) are inserted and clamped in order to secure them against escape from the sleeve. Good clamping is provided by a clamping element (5) separate from the sleeve.

### BACKGROUND

The invention relates to a medical device, in particular, an occlusion instrument, for closing congenital or pathologically formed openings in the human body with a collapsible braid of wires and/or filaments made from Nitinol or a similar material with a shape-memory or memory effect, wherein the ends of the wires or filaments of the braid are brought together and held on at least one closure assembly, with the closure assembly being formed by a sleeve in which the ends are inserted and prevented from escaping from the sleeve by clamping.

Today, such devices are often inserted by cardiac catheter technology for the treatment of heart diseases and are known from, for example, U.S. Pat. No. 5,725,552, U.S. Pat. No. 5,846,261, DE 103 38 702 B3, or DE 695 34 505 T2. The devices can be used to reliably treat openings due to different causes, for example, atrial septal defects, such as a persistent foramen ovale or a so-called defect of the secundum type, through the use of an optimally self-centering device as an occlusion system that is later grown over and around by tissue. Such a medical device is typically introduced into the body via a leg vein, wherein the device is collapsed in a tube and moved by an insertion catheter called a “pusher” in the direction of the heart. After withdrawing the tube, the device unfolds due to the shape memory of its material into the form allocated to it in advance and can be brought into position and secured at its provided location of use.

For the previously mentioned devices from the state of the art, however, the braid made from Nitinol wires or filaments often can be manipulated only with difficulty, because the free ends of the wires must be brought together and secured by a closure assembly and simultaneously a possibility must be offered to connect an insertion catheter to the device. This is made difficult by the fact that Nitinol cannot fuse with steel. Likewise, for example, a thread with the necessary small dimensions can be formed as a connection in the Nitinol only with much difficulty through cutting or material removal.

Securing the wires and filaments to a sleeve generating a clamping effect depends on how good this clamping effect is created during assembly. If the clamping is inadequate, then the device has a corresponding deficiency.

### SUMMARY

Therefore there exists the objective of creating a corresponding medical device that guarantees a secure connection of the free ends of the wires of the braid and that can be manipulated easily and that can be connected detachably and reliably to a positioning means.

This objective is met according to the invention in that the closure assembly is provided with a clamping element that is arranged separate from the sleeve or on the sleeve and that creates a non-positive and/or positive fit connection of the ends of the wires or filaments of the braid to the sleeve and that the clamping element passes through the sleeve and is secured to a holding element on its end facing away from the braid.

The mentioned sleeve consequently surrounds the wire ends that form a press fit at some position of their passage through the sleeve with the adjacent inner wall of the sleeve, in particular, at its edge or front end, wherein, for one, the wire ends are secured on the sleeve with resistance to tensile and/or shear forces and, second, a positioning means for the medical device can be arranged on the sleeve, optionally by another attachment means. The sleeve as a closure assembly and optionally other objects arranged on it can be made from, for example, an implantable, biocompatible stainless steel.

A good clamping effect is achieved between the sleeve and the wire ends, in that the clamping element grips between the wire ends and they are forced away from one another such that a good clamping connection between the wire ends and the sleeve is generated, so that the clamping effect does not have to be realized by a compression of the sleeve itself. This clamping connection is secured by the holding element, because in this way a clamping element separate from the sleeve can be prevented from escaping from its seat within the sleeve.

A preferred and advantageous improvement of the medical device according to the invention provides that the clamping element of the closure assembly is formed by at least one bolt or pin that is essentially conical in some regions and that engages at least partially in the sleeve. Here, the said bolt or pin does not necessarily have to engage as a clamping element with its entire length in the sleeve, but instead can project past the sleeve opening, wherein the conical region of the clamping element is preferably located at least in the region of the inlet opening in the sleeve for the clamping element and thus provides for a clamping of the ends of the braid at this position, especially at the edge of the relevant opening.

Because the mentioned holding element should preferably be well adapted to the shape of the closure assembly, an advantageous improvement can be provided in that the holding element has a disk-like construction and advantageously has the same cross section as the sleeve, in particular, a circular cross section. In this way, the sleeve opening facing the ends of the braid wires can also be optimally closed.

A reliable securing of the clamping element to the holding element is achieved in a simple way in another improvement of the device in which the holding element has passage openings for the end or ends of the clamping element and these ends are connected permanently to the holding element in the region of the output of the passage openings, in particular by a fused connection. In this way, for example, the end or ends of the clamping elements pass completely through the passage openings of the holding element and are worked so that they grip over the edge of the output of each passage opening and are thereby prevented from an opposite movement out of the opening and then are also, for example, fused.

For preventing a possibly harmful influence, exposed wire ends of the braid on the outlet opening of the sleeve can be counteracted with a different improvement of the medical device in which the holding element is connected on its flat side facing the braid with the adjacent front end of the sleeve. As a result, a sleeve that is still open only toward the braid is produced. The mentioned connection between the holding element and the sleeve connection can be realized in an especially simple way by a welded connection.

One embodiment of the invention represents another possibility to implement the clamping connection between the wire ends and sleeve in a simple way in which the sleeve has a different opening cross section on its ends and the opening has a conical profile between the ends. In this embodiment, the clamping is preferably generated at the sleeve opening with the smaller opening cross section that is, in turn, preferably turned toward the wire or filament braid. Here, the clamping can also be supported by a clamping element.

In a way for especially good manipulation, the support is realized by the clamping element in an improvement of the medical device in which the clamping element is introduced into the sleeve from the end of the sleeve with the larger opening cross section and optionally formed in one piece with the holding element. Here, the clamping element can have a conical form over its entire length and can extend to a point in the direction of the wire braid, wherein, in a one-piece configuration with the holding element, this merely has to be secured on the sleeve.

A connection of the medical device to other devices, in particular, positioning means, wherein this connection is independent of the dimensions of the sleeve as a closure assembly for the wire braid and wherein the medical device is to be connected to this positioning means during an insertion phase into the body, can be implemented for an embodiment of the medical device that is inventive by itself in which on the sleeve there can be an especially sleeve-shaped connection piece that engages at least partially over the sleeve. This connection piece in which the sleeve at least partially engages approximately flush when its outer diameter corresponds approximately to the inner diameter of the opening of the connection piece forms a kind of adapter between the closure assembly and the mentioned positioning means formed, for the most part, as an insertion catheter.

In this way, in one improvement it can have one or more attachment means of different types of which one is formed in an especially preferred way by a thread arranged on the closing piece, for example, an Ml thread. Other attachment means, however, are also possible, like a complete elimination of a connection sleeve, wherein then the closure assembly would be provided with attachment means, for example, an external thread. In its construction as a sleeve-shaped connection piece, this is to be permanently connected again to the sleeve of the closure assembly in an especially simple way with a positive-fit connection, for example, by a welded connection. The reliability of the connection is increased more if the predominant part of the sleeve is held in the opening of the connection piece.

One advantageous measure for protecting, for example, a clamping element located between the wire ends located in the sleeve against mechanical influences can be provided in that, in another configuration of the invention, between the sleeve and the wire braid there is a protection element that is formed advantageously as a clamp and whose ends engage at least partially over the sleeve and optionally the connection piece. Securing the protection element acts on the part of the mentioned parts gripped on the outside.

As already indicated in the preceding description, in particular, permanent connections can be created between the individual parts in an especially good way by welded connections, in particular, laser welded connections, which requires, in turn, weldable materials. Therefore, advantageously for a configuration of the medical device, the sleeve and/or the clamping element and/or the holding element and/or the connection piece are formed from a weldable, implantable material, in particular, a stainless steel. Likewise, however, it is conceivable in another configuration of the device that the sleeve is formed from Nitinol and then, for example, the holding element closing a sleeve opening is connected permanently to the connection piece by a welded connection.

In the case of a cylinder-symmetric braid, a configuration of the medical device in which the closure assembly is arranged coaxial to its main axis is especially simple for the manipulation of the device, because it can be easily collapsed and refolded.

### BRIEF DESCRIPTION OF THE DRAWINGS

The invention will be explained in greater detail below with reference to embodiments in the drawings. Shown herein in partially schematic representation are

FIG. 1 is a perspective side view of a medical device in its position of use with closure assembly arranged on its upper end,

FIG. 2 is a sectioned side view of a first embodiment of the closure assembly with a clamping element and a connection piece with attachment means,

FIG. 3 is a view of the closure assembly of FIG. 2 with protection means arranged thereon, and

FIG. 4 is a sectioned side view of another embodiment of the closure assembly in which the sleeve has a conical construction and the clamping element is formed in one piece with the holding element.

### DETAILED DESCRIPTION OF THE PREFERRED EMBODIMENTS

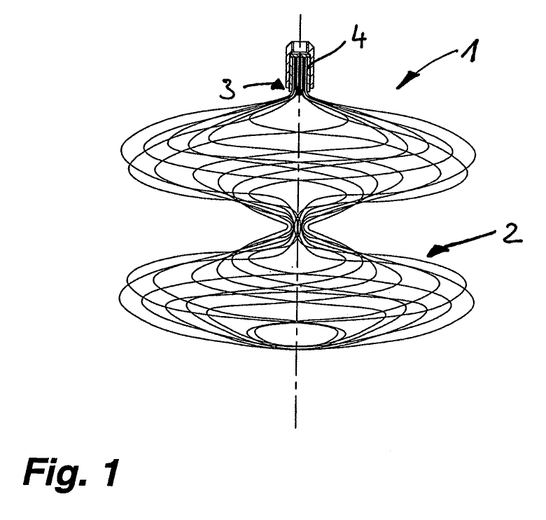

In FIG. 1, a device, a so-called ASD occlusion, designated as a whole with 1 is shown in its position of use. This device can be used for closing congenital or pathologically formed openings in the human body. The medical device 1 has a collapsible braid 2 of wires and/or filaments made from Nitinol or similar material with shape memory or memory effect, so that the braid 2 with elongated wires in a not-shown catheter can be introduced into the human body, wherein the ends 3 of the wires or filaments of the braid are brought together and held by a closure assembly 4.

The closure assembly 4 is formed by a sleeve, here a stainless steel sleeve in which the ends 3 are introduced and secured against escaping from the sleeve by a clamping effect. The closure assembly 4 arranged on the end of the medical device at the top for the viewer has, on its end facing away from the braid 2, a thread as attachment means 9 by which a similarly not-shown positioning means, for example, an insertion catheter, can be arranged on the closure assembly 4 and thus on the described composite device and this device can be moved and guided in this way. After successful introduction of the device 1, e.g., into an atrium of the heart, and subsequent unfolding due to the shape memory of its material in the position of use shown in FIG. 1, the relevant insertion catheter can be withdrawn.

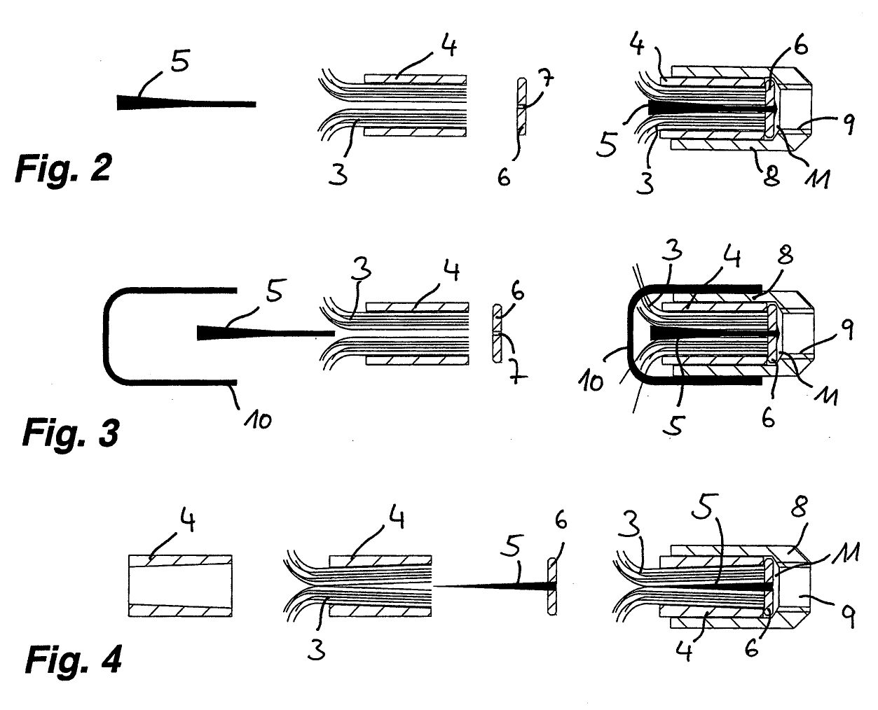

In FIG. 2, a first embodiment of the closure assembly 4 formed by a sleeve is shown in greater detail, wherein this it is provided with a clamping element 5 separate from the sleeve in the form of a pin made from stainless steel. In FIG. 2, first, at the left is the pin and then to the right of this pin is the closure assembly 4 with ends 3 of wires introduced therein and to the right, in turn, of the closure assembly is a disk-shaped holding element 6 made from stainless steel. At the far right in FIG. 2, the closure assembly is shown in assembled state with the connection piece 8 arranged thereon.

It further follows from FIG. 2 that the clamping element 5 is introduced from the left between the ends 3 of the wires in the sleeve opening, with the wire ends emerging from the sleeve and transitioning into the braid. The pin has a conical form in some regions and, in the assembled state shown at the right, the predominant part of the length of the pin engages in the sleeve or passes through the sleeve. In the conical region of the pin, the wires are clamped in an especially strong way in interaction with the inner wall and the edge of the sleeve. The disk-shaped holding element 6 is used for securing the end of the clamping element 5 passing through the sleeve. For this purpose, the holding element 6 that, incidentally, has essentially the same cross section as the sleeve, is itself provided with a passage opening 7 engaged by the end of the clamping element 5 and the end of the clamping element is welded with this holding element 6 in the outlet region of the passage opening 7 facing away from the wire braid 3, so that the clamping element 5 cannot escape from the sleeve in the direction towards the left. The holding element 6 is connected again, in turn, on its flat side facing the wire braid 3 to the adjacent front end of the sleeve, namely by a welded connection.

In the assembled state, on the closure assembly 4 there is a sleeve-shaped connection piece 8 that is provided with an opening 11 in which the closure assembly 4 engages, so that the connection piece 8 partially grips over the sleeve of the closure assembly 4. The connection piece 8 is here connected permanently to the sleeve by a welded connection at contact positions and has, pointing toward the right, attachment means 9 in the form of a thread for the arrangement of a not-shown positioning means for the device 1.

The situation shown in FIG. 3 corresponds to that from FIG. 2 with the exception that, between the sleeve and the wire braid 2, there is protection element 10 that is formed as a clamp and whose ends partially grip over the sleeve and optionally the connection piece 8. The insides of the clamp ends here come to lie on the outsides of the connection piece 8 and are connected again permanently to this, so that, as a whole, the clamp can protect the pin from mechanical influences, while the ends 3 of the wires transitioning to the braid 2 pass by the clamp at the side.

In FIG. 4, another embodiment of the closure assembly 4 is shown in separate parts and in the assembled state in which the sleeve shown at the far left has, on its ends, a different opening cross section and the opening has a conical profile between the ends. When the ends 3 of the wires are inserted into the sleeve in the middle of the diagram, these are already tightened somewhat at the narrower outlet end of the ends 3. Then the clamping element 5 connected in one piece to the holding element 6 is inserted from the sleeve end with greater opening cross section into the sleeve and the holding element is connected again in an already known way to the front end of the sleeve. The holding and connection to the connection piece is also already known from FIGS. 2 and 3.

Consequently, the invention described above relates to a medical device 1, in particular, an occlusion instrument, for closing congenital or pathologically formed openings in the human body with a collapsible braid 2 of wires and/or filaments made from Nitinol or similar material with shape memory or memory effect, wherein the ends 3 of the wires or filaments of the braid are brought together and held by a closure assembly 4. The device can be manipulated in an especially easy way and can be connected to attachment means such that the closure assembly 4 is formed by a sleeve in which the ends 3 are inserted and prevented from escaping from the sleeve by a clamping effect.

Here, a clamping element 5 separate from the sleeve provides a good clamping effect.

1. A medical device (1), for closing congenital or pathologically formed openings in a human body, comprising a collapsible braid (2) of wires or filaments made from Nitinol or a material with shape memory or memory effect, ends (3) of the wires or filaments of the braid are brought together and held by at least one closure assembly (4), the closure assembly (4) comprises a sleeve in which the ends (3) are inserted and prevented from escaping from the sleeve by a clamping effect, and a clamping element (5) that is arranged separate from the sleeve or on the sleeve and that creates a connection of the ends (3) of the wires or filaments of the braid (2) with the sleeve, and the clamping element (5) passes through the sleeve and is secured on a holding element (6) on an end of the sleeve away from the braid (2).

2. The medical device according to claim 1, wherein the clamping element (5) of the closure assembly (4) is formed by at least one bolt or pin that is conical in at least some regions and that engages at least partially in the sleeve.

3. The medical device according to claim 1, wherein the holding element (6) has a disk-like shape and a cross section that is the same as the sleeve.

4. The medical device according to claim 1, wherein the holding element (6) has passage openings (7) for an end or ends of the clamping element (5) and the end or ends are connected permanently to the holding element (6) by a welded connection in a region of an outer side of the passage openings (7).

5. The medical device according to claim 1, wherein the holding element (6) is connected, on a flat side thereof facing the braid (2), to an adjacent front end of the sleeve.

6. The medical device according to claim 1, wherein the sleeve has, on its ends, a different opening cross section and a passage through the sleeve has a conical profile between the ends.

7. The medical device according to claim 6, wherein the clamping element (5) is inserted into the sleeve from the sleeve end with a larger opening cross section.

8. The medical device according to claim 1, further comprising a sleeve-shaped connection piece (8) that engages at least partially over the closure assembly (4).

9. The medical device according to claim 8, wherein the connection piece (8) is connected permanently to the sleeve.

10. The medical device according to claim 8, wherein at least one attachment means (9) is provided on the connection piece (8) for the placement of a positioning element for the device (1).

11. The medical device according to claim 1, wherein between the sleeve and the wire braid (2) there is a protection element (10) formed as a clamp and having ends that at least partially grip over the sleeve.

12. The medical device according to claim 8, wherein at least one of the sleeve or the clamping element (5) or the holding element (6) or the connection piece (8) are formed from a weldable, implantable material.

13. The medical device according to claim 1, wherein the sleeve is made from Nitinol material.

14. The medical device according to claim 1, wherein the collapsible braid comprises a cylinder-symmetric braid (2) and the closure assembly (4) is arranged coaxial to a main axis thereof.

15. The medical device according to claim 3, wherein the cross section is circular.

16. The medical device according to claim 7, wherein the clamping element is formed in one piece with the holding element.

17. The medical device according to claim 9, wherein the connection piece (8) is welded to the sleeve.

18. Medical device according to claim 2, wherein at least one of the sleeve or the clamping element (5) or the holding element (6) or the connection piece (8) are formed of stainless steel.
